(19) United States
(12) Patent Application Publication
(10) Pub. No.: US 2026/0243391 A1
(43) Pub. Date: Aug. 20, 2026

(54) SPRING ARM DEVICE AND SUPPORT ARM SYSTEM FOR HOLDING A MEDICAL APPLIANCE AND/OR LIGHTING APPLIANCE

(71) Applicant: ONDAL MEDICAL SYSTEMS GMBH, Hünfeld (DE)

(72) Inventor: Markus ABEL, Fulda (DE)

(21) Appl. No.: 19/156,040

(22) PCT Filed: Feb. 8, 2024
(86) PCT No.: PCT/EP2024/053260

§ 371 Date: Aug. 13, 2025

(30) Foreign Application Priority Data
Feb. 15, 2023 (EP) 23156876.7

(51) Int. Cl. F16M 13/02; A61B 90/35; A61B 90/50; F16M 11/10; F16M 11/20; F21V 21/28

(52) U.S. Cl. CPC F16M 13/022; A61B 90/35; A61B 90/50; F16M 11/10; F16M 11/2014; F21V 21/28

(57) ABSTRACT

The present invention relates to a spring arm device (1) for a support arm system (100) for holding a medical appliance and/or lighting appliance, comprising a spring tube (3) arranged on a fastening unit (5) for rotation about a first axis of rotation (21), a spring rod (4) arranged at least partially in the interior of the spring tube (3) and displaceable relative to the spring tube (3) in the longitudinal direction (2) of the spring tube (3), and a lever element (6) which is connected to the spring rod (4) for rotation about a second axis of rotation (22) oriented parallel to the first axis of rotation (21) and which is arranged, at its end opposite the spring rod (4), on the fastening unit (5) for rotation about a third axis of rotation (23) which is spaced apart from the first axis of rotation (21) and oriented parallel to the first axis of rotation (21), wherein the lever element (6) is arranged radially outside the spring tube (3) in relation to the longitudinal direction (2) of the spring tube (3). The invention also relates to a support arm system (100), comprising such a spring arm device (1), for holding a medical appliance and/or lighting appliance.

### TECHNICAL FIELD

The present invention relates to a spring arm device for a support arm system for holding a medical appliance and/or a lighting appliance, and to a support arm system for holding a medical appliance and/or a lighting appliance.

### PRIOR ART

Support arm systems are used in operating theatres, for example, in order to hold medical apparatus or a medical appliance in a spatially displaceable manner. As an example, to this end, a support arm can be movably anchored to a ceiling, a wall or a floor by means of a bearing assembly. Mounting on a frame with castors is also possible. A support arm system which is designed to hold medical appliances can also be designated as a medical support system. Support arm systems usually have at least one axis of movement, so that the medical appliance which is held, which can also be designated as a technical medical appliance and, for example, may have a lighting appliance such as an OR light, a monitor, examination appliances, one or more injectors, OR instruments or dental drills, can be moved as required. The bearing which enables the movement can also be designated as a joint device or support arm joint device, wherein these may also comprise a support arm. This means that the medical appliance can be brought into a comfortable and secure position for Carrying out the respective medical procedures and investigations. The spatial displacement of medical appliances is essential in many medical procedures in order to facilitate the work of medical personnel, or indeed to make it possible.

By swivelling a support arm, however, a lever arm relating to its bearing can be varied. In particular, when the support arm is swivelled about a substantially horizontal axis, whereupon the height of the medical appliance held in it can be varied, a lever arm of the weight of the medical appliance is varied with respect to the mounting or bearing of the support arm. Nevertheless, it is still desirable for the medical appliance to remain in the position set by swivelling the support arm and for an adjustment of the position of the medical appliance, in particular against the force of gravity, to be possible for a user to make without expending too much force.

To this end, spring-balanced support arms, primarily as spring arms or spring arm devices, are often used. In this regard, two arm elements are provided which are movable relative to each other and are mutually supported by means of a spring. Swivelling the spring arm varies the lever arm of the spring force corresponding to the lever arm of the load, whereupon the medical appliance can be held equally well in different positions. As an alternative or in addition, the spring may also be compressed or stretched by swivelling, whereupon a corresponding supporting force can be varied.

Support arm systems and spring arm devices of this type are known, for example, from EP 1 478 872 A1. As a rule, spring arm devices comprise a spring tube which is disposed on a mounting unit for rotation about a largely horizontal axis of rotation, a spring rod which is disposed at least partially in the interior of the spring tube and is displaceable with respect to the spring tube, and a lever element one end of which is rotatably connected to the spring rod in the interior of the spring tube and the other end of which is rotatably disposed on the mounting unit.

### DISCLOSURE OF THE INVENTION

Starting from the known prior art, an objective of the present invention is to provide an improved spring arm device for a support arm system for holding a medical appliance and/or a lighting appliance, as well as a Corresponding support arm system for holding a medical appliance and/or a lighting appliance. In particular, a spring arm device with a comparatively large range of movement should be provided.

The objective is achieved by a spring arm device for a support arm system for holding a medical appliance and/or a lighting appliance with the features of claim 1. Advantageous further embodiments are described in the dependent claims, the description and the figures.

Accordingly, a spring arm device for a support arm system for holding a medical appliance and/or a lighting appliance is proposed, comprising a spring tube disposed on a mounting unit for rotation about a first axis of rotation, a spring rod disposed at least partially in the interior of the spring tube and displaceable relative to the spring tube in the longitudinal direction of the spring tube, and a lever element which is connected to the spring rod for rotation about a second axis of rotation orientated parallel to the first axis of rotation and which, at its end opposite to the spring rod, is disposed on the mounting unit for rotation about a third axis of rotation which is spaced apart from the first axis of rotation and orientated parallel to the first axis of rotation.

In the proposed spring arm device, the lever element is disposed radially outside the spring tube with respect to the longitudinal direction of the spring tube. In this manner, in contrast to prior art spring arm devices in which the lever element extends inside the spring tube and is connected to the spring rod therein and the range of movement is restricted by the collision points between the lever element and the inside of the spring tube, the swivelling range of the spring tube about the first axis of rotation is not restricted as regards its range of movement. Moreover, disposing the lever element radially outside the spring tube means that the spring tube and the lever element can swivel substantially freely relative to each other without leading to a collision of the lever element and spring tube. The possible swivelling angle of the spring tube about the first axis of rotation is consequently substantially only restricted by the connection of the spring tube to the mounting unit, but is not restricted by the lever element. Correspondingly, the spring arm device in accordance with the invention has a substantially enhanced swivelling range about the first axis of rotation compared with conventional spring arm devices of the prior art.

The spring arm device may be a component of an operating theatre support arm system. As the spring arm device of an operating theatre support arm system, it is consequently configured and constructed for holding medical equipment or a medical appliance, for example a medical lighting appliance, in a spatially displaceable manner.

The term “rotatable bearing” in this document should in particular be understood to mean that exclusively a. rotational movement of the components of the rotatable bearing relative to one another about the respective axis of rotation is possible. In particular, compressive and tensile forces as well as torques which do not act about the axis of rotation are transmitted by the rotatable bearing.

Preferably, the spring rod and the lever element are connected to each other via a connecting piece. The connecting piece is preferably connected to the spring rod radially inside the spring tube, consequently in general in the interior of the spring tube, and to the lever element radially outside the spring tube. The connecting piece preferably extends in the direction of the second axis of rotation and/or preferably through a recess provided in the spring tube from the interior of the spring tube to radially outside the spring tube.

The term “radially inside” should be understood here to mean that region which, with respect to the longitudinal direction of the spring tube, has an extent transversely to the longitudinal direction which is less than or equal to the internal radius of the spring tube or its inner surface in the case of non-circular spring tubes.

The term “radially outside” describes that region which, with respect to the longitudinal direction of the spring tube, has an extent transversely to the longitudinal direction which is larger than the external radius of the spring tube or its outer surface in the case of non-circular spring tubes.

It has been shown to be advantageous for the recess to be an elongated hole orientated in the longitudinal direction, wherein the connecting piece is movable along the elongated hole in the longitudinal direction.

In accordance with a preferred embodiment, in the direction of the first axis of rotation, the lever element is at a predetermined distance from the spring tube, in particular from a connection or a connecting region of the spring tube for connection to the mounting unit. This can ensure that the lever element and the spring tube do not collide when the spring tube swivels about the first axis of rotation.

When the spring rod is configured as a hollow rod, at least one supply channel, for example a supply of medium, such as compressed air, oxygen or nitrogen, and/or a supply of power and/or data, for a medical appliance and/or a lighting appliance attached to the spring arm device, can be disposed through the interior of the hollow rod.

In accordance with a preferred embodiment, the mounting unit comprises a joint section in which the spring tube and the lever element are connected to the mounting unit, and furthermore a connecting section, preferably constructed as a journal, for connecting the mounting unit to a load-bearing structure, preferably a ceiling, a wall or a floor of a room, or a support frame or support arm, wherein, in the direction of the first axis of rotation, the joint section and the connecting section are at a predetermined distance with respect to each other. By means of this spatial separation or distancing of the connecting section and joint section, the spring arm device and in particular a housing for enclosing at least the joint section can be of comparatively simple construction. Expressed another way, with respect to a plane orientated perpendicular to the first axis of rotation, the joint section lies on a first side of the plane and the connecting section lies on the other side of the plane. The spring tube and the lever element and their connections consequently lie completely and exclusively on the joint section side. This means that the spring tube can be moved relative to the connecting section past the connecting section without colliding with it. This is constructively achieved by means of the predetermined distance between the joint section and connecting section in the direction of the first axis of rotation. expressed another way, the predetermined distance exists between first plane orientated perpendicular to the first axis of rotation and a second plane orientated perpendicular to the first axis of rotation. The first plane is defined by the point of the joint section, including the connected spring tube, lying closest to the connecting section. Correspondingly, the second plane is defined by the point of the connecting section lying closest to the joint section.

Advantageously, the lever element is disposed opposite the connecting section with respect to the spring tube.

Preferably, a first housing part is provided which is preferably connected to the spring tube in a manner which is fixed against rotation and which encloses the joint section. In addition, preferably, a second housing part is provided which is preferably connected to the connecting section in manner which is fixed against rotation, which preferably encloses at least a portion of the connecting section. A separating plane between the first housing part and the second housing part may be orientated perpendicular to the first axis of rotation and—viewed in the direction of the first axis of rotation—may be disposed in a region between the joint section and the connecting section. Expressed another way, with respect to the exactly one separating plane orientated perpendicular to the first axis of rotation, the first housing part lies on a first side of the exactly one separating plane and the second housing part lies on the other side of the separating plane. Consequently, the first housing part lies completely and exclusively on the joint section side and the second housing part consequently lies completely and exclusively on the connecting section side. The separating plane may be designated as or be coincident with the aforementioned plane orientated perpendicular to the first axis of rotation, the joint section being disposed on the first side thereof and the connecting section being disposed on the other side. In principle, the separating plane may be any plane which is perpendicular to the first axis of rotation between the aforementioned first plane and the second plane.

A seal for sealing the first housing part and the second housing part against the environment may also be disposed at the level of this separating plane.

In accordance with a further preferred embodiment, the spring rod is mounted for translation in the spring tube, preferably slide-mounted, wherein preferably, the spring rod comprises at least one carriage which is mounted, preferably slide-mounted, for translation with respect to the spring tube. Preferably, on a first side, the spring rod comprises a first carriage which is mounted for translation with respect to the spring tube and on a second side, the spring rod comprises a second carriage which is mounted for translation with respect to the spring tube.

Advantageously, the spring rod has a maximum external diameter which is smaller than a minimum internal diameter of the spring tube at least in the region of the range of movement of the spring rod in the spring tube. Advantageously, the at least one carriage positions the spring rod at a distance from the spring rod, preferably concentrically. Between the maximum external diameter of the spring rod and the inner wall of the spring tube there is consequently a distance of a predetermined magnitude. Expressed another way, the spring rod is preferably at a distance from the inner wall of the spring tube in the spring tube.

The spring rod may be pre-tensioned relative to the spring tube by a spring element for pre-tensioning the spring rod relative to the spring tube. In particular, the spring element may be a compression spring which is disposed in the interior of the spring tube and extends radially outside and around the spring rod. Expressed another way, the spring rod may pass through the compression spring. Expressed yet another way, the spring element, for example in the form of the compression spring, may advantageously be configured as a coil spring pushed onto the spring rod.

The spring element may be stopped at one end by a stop on the spring tube and at its other end by a stop on the spring rod.

In accordance with a preferred embodiment, on a side opposite to the mounting unit with respect to the spring element, a carriage, for example the aforementioned first or second carriage, may constitute a stop for the spring element on the spring rod. Consequently, the spring element can be braced on the spring rod in the direction of the longitudinal direction of the spring rod or the spring tube via the carriage. Furthermore, the other end of the spring element may be braced on a stop on the spring tube, for example in the form of an indentation in the spring tube. When a distance in the longitudinal direction between the two stops is smaller than a neutral length of the spring element, which is the case in an unstressed state, the spring element can be pre-tensioned.

A level of the pre-tension provided by the spring element may be adjusted via an optional alignment unit which may be disposed on the mounting unit. The alignment unit may, for example, be configured to vary the distance of the third axis of rotation relative to the first axis of rotation.

The aforementioned objective is furthermore achieved by means of a support arm system for holding at least one medical appliance and/or a lighting appliance with the features of claim 9. Advantageous further embodiments will become apparent from the present description and the figures.

Accordingly, a support arm system for holding at least one medical appliance and/or a lighting appliance is proposed which comprises at least one spring arm device in accordance with one of the present embodiments.

The advantages and effects described with respect to the spring arm device are also obtained by means of the support arm system, and so a repeated description is dispensed with here in the interests of avoiding redundancies.

### BRIEF DESCRIPTION OF THE FIGURES

Preferred further embodiments of the invention will be described in more detail in the description below of the figures, in which:

FIG. 1 diagrammatically shows a perspective side view of a support arm system with a spring arm device;

FIG. 2 diagrammatically shows a further perspective side view of the spring arm device of FIG. 1;

FIG. 3 diagrammatically shows a perspective side view of subsection of the support arm system of FIG. 1;

FIG. 4 diagrammatically shows a further perspective side view of the subsection of FIG. 3;

FIG. 5 diagrammatically shows view Of a swivelling range of a spring tube of the spring arm device about a first axis of rotation; and

FIG. 6 diagrammatically shows a perspective side view of the spring arm device of FIG. 1 with a point cloud for a holding region for the spring tube for holding an appliance.

### DETAILED DESCRIPTION OF PREFERRED EXEMPLARY EMBODIMENTS

Preferred exemplary embodiments will be described below with the aid of the figures. In this regard, identical or similar elements or elements which have the same action will be provided with identical reference symbols in the various figures and a repeated description of these elements will be partially dispensed with in the interests of avoiding redundancies.

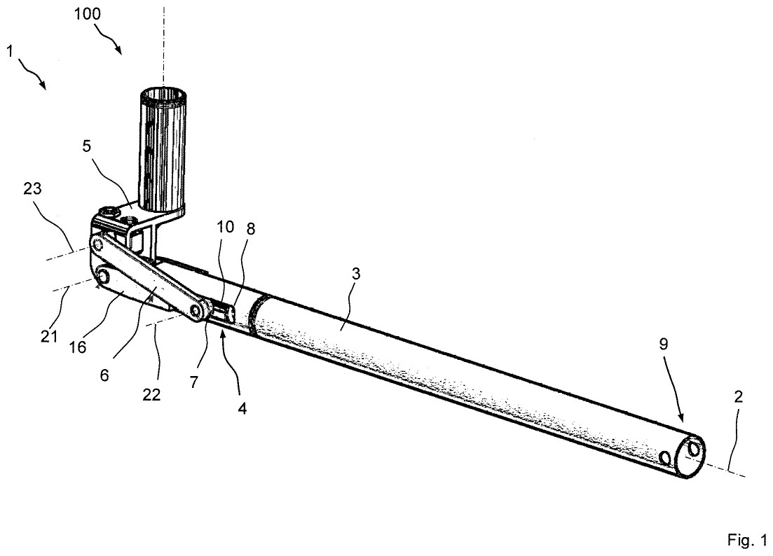

FIG. 1 diagrammatically shows a perspective side view of a support arm system 100 with a spring arm device 1. The support arm system 100 is configured to hold a medical appliance (not shown) or, alternatively, a lighting appliance.

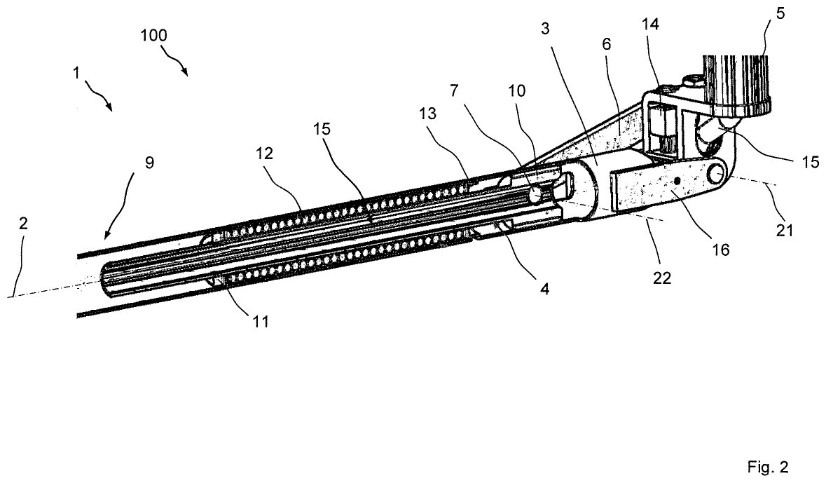

The spring arm device 1 comprises a spring tube 3 which extends in a longitudinal direction 2. A spring rod 4 which is displaceable in the longitudinal direction 2 relative to the spring tube 3 is disposed inside the spring tube 3, as can be seen in more detail in FIG. 2. On a first side, the spring tube 3 is rotatably disposed on a mounting unit 5 about a substantially horizontally orientated first axis of rotation 21, shown in a fitted state of the spring arm device at the deployment location.

Furthermore, the spring arm device 1 comprises a lever element which is connected to the spring rod 4 for rotation about a second axis of rotation 22 which is orientated parallel to the first axis of rotation 21 and on its side opposite to the spring rod 4, it is disposed on the mounting unit 5 for rotation about a third axis of rotation 23 which is orientated parallel to the first axis of rotation 21 and at a distance from the first axis of rotation 21.

As can be seen in FIG. 1, the lever element 6 is disposed radially outside the spring tube 3 with respect to the longitudinal direction 2.

The spring rod 4 and the lever element 6 are connected to each other via a connecting piece 7 which is connected to the spring rod 4 radially inside, i.e. in the interior of the spring tube 3 and to the lever element 6 radially outside the spring tube 3. Here, the connecting piece 7 extends through a recess 8 in the form of an elongated hole extending in the longitudinal direction 2 provided in the spring tube 3.

At its end opposite to the mounting unit 5, the spring tube 3 has a holding section 9 in the form of a connection for connecting an appliance to be held. The term “holding” of an appliance should be understood here to mean that the appliance to be held is connected to the holding section 9, wherein the weight of the appliance on the holding section is transferred into the spring tube 3. The term “holding” here also encompasses the fact that a further support arm, for example a further spring arm, or a holding device may be disposed between the appliance and the spring tube.

FIG. 2 diagrammatically shows a perspective side view of the spring arm device of FIG. 1 from a different viewing direction, in which a portion of the spring tube 3 is cut away in order to provide a view into the interior of the spring tube 3.

The spring rod 4 extends in the longitudinal direction 2 inside the spring tube 3. At its end facing the mounting unit 5, it comprises a first carriage 10 which is slidably mounted in the spring tube 3 for translation in the longitudinal direction 2. The connecting element 7 connected to the spring rod 4 extends in the region of the first carriage 10.

At its opposite side, the spring rod 4 comprises a second carriage 11 which is slidably mounted for translation with respect to the spring tube 3.

On the side of the spring rod 4 opposite to the mounting unit 5, the second carriage 11 forms a stop for a spring element 12, formed here as a compression spring, for pre-tensioning the spring rod 4 relative to the spring tube 3. On the other side, the spring element 12 is braced in an indentation 13 in the spring tube 3. The distance in the longitudinal direction 2 between the second carriage 11 and the indentation 13 is smaller than a neutral length of the spring element 12 which it has in an unstressed state. The spring element 12 is consequently pre-tensioned.

The level of the pre-tension can be adjusted via an alignment unit 14 on the mounting unit 5. The alignment unit 14 enables the distance of the third axis of rotation 23 relative to the first axis of rotation 21 to be varied. The pre-tension is essentially dependent on the lever length, i.e. the distance between the first axis of rotation 21 and the third axis of rotation 23.

The reference symbol 15 indicates a power cable which extends from the mounting unit 5 through the spring rod 4, constructed as a hollow rod, to the holding section 9.

It will be appreciated from viewing FIGS. 1 and 2 in combination that the spring tube 3 comprises a fork-shaped connecting region 16 for connection to the mounting unit 5.

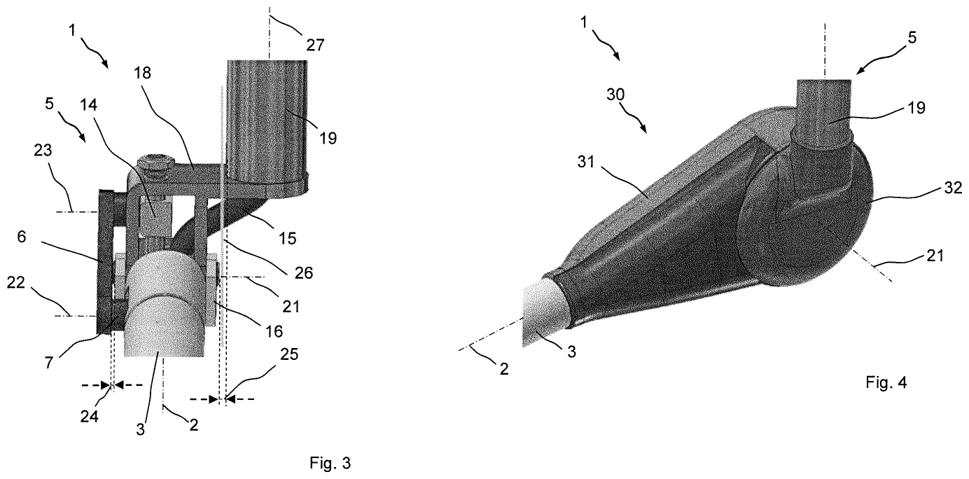

FIG. 3 diagrammatically shows a side view of a subsection of the spring arm device 1 of FIG. 1.

This view shows that in the direction of the first axis of rotation 21, the lever element 6 is at a predetermined distance 24 from the spring tube 3, more precisely from the connection of the spring tube 3 to the mounting unit 5. Correspondingly, it is possible for the lever element 6 and the spring tube 3 to move past one another without colliding.

The mounting unit 5 here is divided into a joint section 18, in which the spring tube 3 and the lever element 6 are connected to the mounting unit 5, and a connecting section 19 connected to the joint section 18 and configured as a journal for connecting the mounting unit 5 to a load-bearing structure, for example a ceiling, a wall or a floor of a room, or a support frame of support arm.

Viewed in the direction of the first axis of rotation 21, the joint section 18 and the connecting section 19 are at a predetermined distance 25 with respect to each other. This enables a separating plane 26 for a two-part housing (see FIG. 4) which is orientated perpendicular to the first axis of rotation 21—viewed in the direction of the first axis of rotation 21—to be provided between the joint section 18 and the connecting section 19.

Expressed another way, with respect to exactly one plane orientated perpendicular to the first axis of rotation, constituted here by the separating plane 26, the joint section 18 lies on a first side of the separating plane 26 and the connecting section 19 lies on the other side of the separating plane 26. The spring tube 3 and the lever element 6 and their connections consequently also lie completely and exclusively on the side of the joint section 18.

FIG. 4 diagrammatically shows a perspective side view of the subsection of FIG. 3, wherein here, a two-part housing 30 is also shown.

The housing 30 seals off the joint section 18 as well as the passage for the power cable 15 in the region of the connecting section 19 against the environment.

The housing 30 comprises a first housing part 31 which encloses the joint section and is connected to the spring tube 3 in a manner which is fixed against rotation, and a second housing part 32 which is connected to the connecting section 19 in a manner which is fixed against rotation and which encloses a lower portion of the connecting section 19. The first housing part 31 can be swivelled about the first axis of rotation 21 relative to the second housing part 32, wherein the first housing part follows the movements of the spring tube 3.

As already described with respect to FIG. 3, the separating plane 26 lies between the first housing part 31 and the second housing part 32 in a region between the joint section 18 and the connecting section 19 viewed in the direction of the first axis of rotation 21. A seal for Sealing the first housing part 31 and the second housing part 32 against the environment is disposed at the level of the separating plane 26. Here, the seal is constructed as a shaft-hub connection with a clearance fit between the housing parts 31, 32. The second housing part 32 comprises a straight cylindrical journal section, not visible in FIG. 4, which extends about the first axis of rotation 21 and which is accommodated in a straight cylindrical hub section which extends about the first axis of rotation 21. The radially outer surface of the journal section and the radially inner surface of the hub section have a predetermined clearance fit.

Expressed another way, with respect to the exactly one separating plane 26 orientated perpendicular to the first axis of rotation 21, the first housing part 31 lies on a first side of the exactly one separating plane 26 and the second housing part 32 lies on the other side of the exactly one separating plane 26.

As an alternative or in addition, the seal may also comprise a labyrinth seal and/or a sealing element, for example a sealing ring.

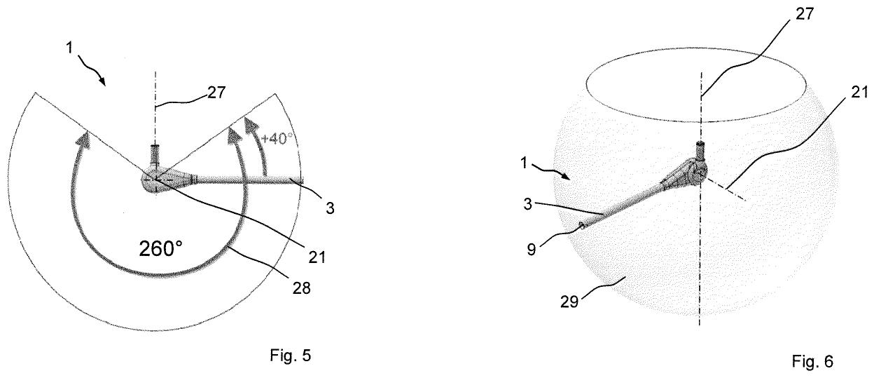

FIG. 5 diagrammatically shows a representation of a swivelling range of the spring tube 3 of the spring arm device 1 of FIG. 1 about the first axis of rotation 21. The first axis of rotation 21 here is substantially horizontal in orientation. As can be seen in FIG. 5, from a horizontal position as shown in FIG. 5, the spring tube 3 covers a swivelling range of 40° against the force of gravity, i.e. upwards. The spring tube 3 can swivel downwards past the vertical so that the swivelling range from the horizontal against the force of gravity is also 40° on the other side.

Overall, in the spring arm device 1, a swivelling angle 28 of the spring tube 3 about the first axis of rotation 21 is optionally 260°.

The swivelling ranges mentioned here of 40° and 260° are only by way of example and the spring arm device is not limited to these swivelling ranges. They may have other values, depending on the application and requirements. In addition, the swivelling range with respect to the vertical may be different on one side compared to the other side.

FIG. 6 diagrammatically shows a perspective side view of the spring arm device 1 of FIG. 5 with a point cloud 29 for the holding region 9 of the spring tube 3. The spring arm device 1 can be swivelled about a vertical axis of rotation 27. This results in the point cloud 29, i.e. the sum of all points which can be reached by the holding region 9 on the basis of the range of movement of the spring arm device 1, namely a segment of a sphere which is composed of the swivelling range from FIG. 5 rotated about the vertical axis of rotation 27.

Insofar as it is applicable, all individual features which are shown in the exemplary embodiments may be combined together and/or exchanged without departing from the scope of the invention.

### LIST OF REFERENCE SYMBOLS

1 spring arm device
2 longitudinal direction
3 spring tube
4 spring rod
5 mounting unit
6 lever element
7 connecting piece
8 recess
9 holding section
10 first carriage
11 second carriage
12 spring element
13 indentation
14 alignment unit
15 power cable
16 connecting region
18 joint section
19 connecting section
21 first axis of rotation
22 second axis of rotation
23 third axis of rotation
24 distance
25 distance
26 separating plane
27 axis of rotation
28 swivelling angle
29 point cloud
30 housing
31 first housing part
32 second housing part
100 support arm system

1. A spring arm device for a support arm system for holding a medical appliance and/or a lighting appliance, comprising:
a spring tube disposed on a mounting unit for rotation about a first axis of rotation, a spring rod disposed at least partially in an interior of the spring tube and displaceable relative to the spring tube in a longitudinal direction of the spring tube, and
a lever element which is connected to the spring rod for rotation about a second axis of rotation orientated parallel to the first axis of rotation and which, at its end opposite to the spring rod, is disposed on the mounting unit for rotation about a third axis of rotation which is spaced apart from the first axis of rotation and orientated parallel to the first axis of rotation, wherein the lever element is disposed radially outside the spring tube with respect to the longitudinal direction of the spring tube.

2. The spring arm device of claim 1, wherein the spring rod and the lever element are connected to each other via a connecting piece, wherein the connecting piece is connected to the spring rod radially inside the spring tube and to the lever element radially outside the spring tube.

3. The spring arm device of claim 2, wherein the connecting piece extends through a recess provided in the spring tube.

4. The spring arm device of claim 3, wherein the recess is an elongated hole orientated in the longitudinal direction, wherein the connecting piece is movable along the elongated hole in the longitudinal direction.

5. The spring arm device of claim 1, wherein in the direction of the first axis of rotation, the lever element is at a predetermined distance from the spring tube, in particular from a connecting region connecting the spring tube to the mounting unit.

6. The spring arm device of claim 1, wherein the spring rod is configured as a hollow rod.

7. The spring arm device of claim 1, wherein the mounting unit comprises a joint section in which the spring tube and the lever element are connected to the mounting unit, and a connecting section for connecting the mounting unit to a load-bearing structure, preferably a ceiling, a wall or a floor of a room, or a support frame or support arm, wherein, in the direction of the first axis of rotation, the joint section and the connecting section are at a predetermined distance with respect to each other.

8. The spring arm device of claim 7, wherein the connecting section is configured as a journal.

9. The spring arm device of claim 7, wherein a first housing part connected to the spring tube encloses the joint section and a second housing part connected to the connecting section encloses at least a portion of the connecting section, wherein a separating plane between the first housing part and the second housing part is orientated perpendicular to the first axis of rotation in a region between the joint section and the connecting section, viewed in the direction of the first axis of rotation.

10. The spring arm device of claim 9, wherein a seal for sealing the first housing part and the second housing part against an environment is disposed at a level of the separating plane.

11. The spring arm device of claim 9, wherein the first housing part is connected to the spring tube in a manner which is fixed against rotation, and/or the second housing part is connected to the connecting section in a manner which is fixed against rotation.

12. The spring arm device of claim 1, wherein the spring rod is mounted for translation in the spring tube, preferably slide-mounted.

13. The spring arm device of claim 12, wherein the spring rod comprises at least one carriage which is mounted, preferably slide-mounted, for translation with respect to the spring tube.

14. The spring arm device of claim 12, wherein on a first side, the spring rod comprises a first carriage which is mounted for translation with respect to the spring tube and on a second side, the spring rod comprises a second carriage which is mounted for translation with respect to the spring tube.

15. A support arm system for holding at least one medical appliance and/or a lighting appliance, comprising at least one spring arm device of claim 1.
